(19) United States
(12) Patent Application Publication
(10) Pub. No.: US 2023/0390180 A1
(43) Pub. Date: Dec. 7, 2023

(54) None

(71) Applicant: CAPSUM, MARSEILLE (FR)

(72) Inventors: Mathieu GOUTAYER, Saint-Malo (FR); Mathieu JACQUOT, Trets (FR)

(21) Appl. No.: 18/323,774

(22) Filed: May 25, 2023

(30) Foreign Application Priority Data
May 30, 2022 (FR) 22 05167

(51) Int. Cl. A61K 8/88; C08G 69/26; C08L 83/08; C08J 3/07; A61K 8/898; A61Q 1/00

(52) U.S. Cl. CPC A61K 8/88; C08G 69/26; C08L 83/08; C08J 3/07; A61K 8/898; A61Q 1/00; A61K 2800/548; C08L 2207/53; C08J 2383/08; C08J 2477/06; A61K 2800/5426; A61K 2800/5424; C08L 2201/54

(57) ABSTRACT

The present invention concerns a dispersion formed of a dispersed phase in the form of drops and a continuous phase, comprising a fatty phase and an aqueous phase non-miscible with each other at ambient temperature and atmospheric pressure, one of the phases among the fatty phase and the aqueous phase being the dispersed phase and the other phase among the fatty phase and aqueous phase being the continuous phase, wherein the drops comprise at least one core and at least one shell formed of at least one anionic polymer and at least one cationic polymer, the cationic polymer being a compound having the following formula (I):

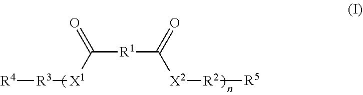

where:
   n is an integer of 1 to 5;
   X¹ and X², the same or different, are —O—, —CH₂— or —NH—;
   R¹, R² and R³, the same or different, are each independently a divalent alkylene radical having 20 to 50 carbon atoms;
   R⁴ and R⁵, the same or different, are each independently H, OH or NH₂, at least one of the groups R⁴ and R⁵ being NH₂.

The subject of the present invention concerns dispersions of drops containing an anionic polymer and a cationic (di)amine polymer. It also concerns compositions, cosmetic compositions in particular, containing said dispersions and the methods for preparing said dispersions.

Amodimethicone, also known as polydimethylsiloxane, is a silicon-based synthetic polymer (or silicone compound). Amodimethicone is a raw material used in cosmetics and particularly for capillary uses. It allows the forming of a sheath around capillary fibre, forming a thin film around hair shafts and on skin, this being desirable for better adhesion of the product, and it is also used for treated dyed hair since it affords protection and maintains colour radiance.

The Applicant uses amodimethicone as lipophilic precursor polymer of coacervates to stabilize dispersions, in particular macroscopic dispersions as described in WO2012120043.

However, in cosmetics, there is ascertained an increasingly strong demand by consumers for compositions free of silicone compounds on account of their environmental impact, since they are non-biodegradable, and/or their suspected health hazard.

There is also ascertained an increasingly strong consumer demand for cosmetic compositions comprising biosourced ingredients. This trend is particularly seen with the arrival of numerous labels such as “Cosmebio”, “Natrue”, “Cosmos” or “Ecocert”, and with the ISO 16128 standard on technical definitions and criteria applicable to natural and organic cosmetic ingredients and products.

There is therefore a need for a non-silicone and biosourced alternative to amodimethicone that is dedicated to the kinetic stability of dispersions, and in particular macroscopic dispersions.

In particular there is also a need for a non-silicone alternative to amodimethicone which ensures the formation of dispersions, macroscopic dispersions in particular, having properties that are at least similar in particular with regard to phenomena of drop aggregation, adhesion of drops to packaging and/or defects of sphericity of drops in dispersions, in particular in macroscopic dispersions. It is therefore the objective of the present invention to provide stable dispersions comprising compounds that are ideally biosourced or prepared from biosourced products, to replace silicone compounds and amodimethicone in particular.

It is a further objective of the invention to provide stable dispersions comprising a biosourced cationic polymer to replace amodimethicone.

As follows from the examples below, the inventors have shown that a compound of formula (I) according to the invention allows the production of dispersions, especially macroscopic dispersions, having satisfactory properties in terms of mechanical strength and kinetic stability.

The present invention therefore concerns a dispersion formed of a dispersed phase in the form of drops and a continuous phase, comprising a fatty phase and an aqueous phase that are non-miscible with each other at ambient temperature and atmospheric pressure, one of the phases from among the fatty phase and aqueous phase being the dispersed phase and the other phase from among the fatty phase and aqueous phase being the continuous phase, wherein the drops comprise at least one core and at least one shell formed of at least one anionic polymer and at least one cationic polymer, the cation polymer being a compound having the following formula (I):

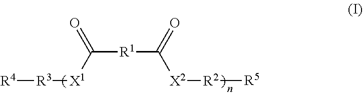

where:
n is an integer of 1 to 5, preferably of 1 to 3, more preferably of 1 or 2;
X¹ and X², the same or different, are each independently —O—, —CH₂— or —NH—, preferably at least one of the groups X¹ and X², even X¹ and X², being —NH—;
R¹, R² and R³, the same or different, are each independently a linear, cyclic or branched, divalent alkylene radical having 20 to 50 carbon atoms, preferably 25 to 45 carbon atoms, better still 30 to 40 carbon atoms, and more preferably 34 to 36 carbon atoms, R¹, R² and R³ possibly including a cycloalkylene radical having 3 to 10 carbon atoms, preferably having 6 carbon atoms, said cycloalkylene radical optionally being substituted by one or more alkyl chains having 6 to 10 carbon atoms,
R⁴ and R⁵, the same or different, are each independently H, OH or NH₂, at least one of the groups R⁴ and R⁵ being NH₂.

In the present invention, the drops of the dispersed phase can indifferently be designated by the term «drops (G1)».

In the present invention, the aforementioned dispersions can indifferently be designated by the term «emulsions».

Dispersion

The dispersion of the invention has the advantage of being kinetically stable in particular over time and when being transported. By «stable» or «kinetic stability», it is meant to designate in the meaning of the present invention, at ambient temperature and atmospheric pressure, the absence of opacification of the aqueous phase, the non-aggregation of the drops with each other, and in particular the absence of coalescence or Oswald ripening of the drops, the non-adhesion of the drops to packaging and the non-leakage of materials from the dispersed phase towards the continuous phase, or vice versa, over a period of time of 1 month or longer, preferably of 3 months or longer, and better still of 6 months or longer.

In one embodiment, a dispersion of the invention comprises less than 0.5%, preferably less than 0.25%, and in particular less than 0.05% by weight of surfactant(s) relative to the total weight of the dispersion, and preferably does not contain any surfactant.

In a first embodiment, a dispersion of the invention is a single emulsion i.e. solely containing an aqueous phase and a fatty phase. Having regard to the type of the phases, a dispersion of the invention can be an emulsion of oil-in-water type (or direct emulsion) or of water-in-oil type (invert emulsion).

In a first embodiment, in the dispersions of the invention, the fatty phase is the continuous phase and the aqueous phase is the dispersed phase.

In a second embodiment, in the dispersion of the invention, the fatty phase is the dispersed phase and the aqueous phase is the continuous phase, preferably in gel form.

In another embodiment, a dispersion of the invention is a multiple emulsion, in particular a double emulsion for example of the type water-in-oil-in-water, oil-in-water-in-oil, or oil-in-oil-in-water.

The fatty phase and the aqueous phase of a dispersion of the invention are non-miscible with each other at ambient temperature and atmospheric pressure. Therefore, the solubility of the fatty phase in the aqueous phase is advantageously lower than 5% by weight, and conversely.

Pairs of oils non-miscible with each are notably described in application FR3063893.

Irrespective of the embodiment, a dispersion of the invention can be termed a macroscopically non-homogeneous mixture of at least two non-miscible phases. In other words, in a dispersion of the invention, the continuous phase can be distinguished from the dispersed phase and conversely, in particular with the naked eye.

The drops G1 of the dispersed phase can be:
single-phase; or
multiphase, in which the drops G1 comprise an intermediate drop of an intermediate phase, the intermediate phase being placed in contact with the shell, and at least one, preferably only one, inner drop of an inner phase arranged in the intermediate drop.

In the present invention, the drops advantageously have apparent monodispersity (i.e. they are seen by the eye as spheres having the same diameter). The drops are advantageously substantially spherical.

The drops of the dispersion of the invention are advantageously macroscopic drops. By «macroscopic», or «macroscopic drop» it is meant to designate, in the present invention, drops of dispersed phase visible to the naked eye as opposed to microscopic drops not visible to the naked eye.

In one embodiment the mean diameter of the drops of the dispersed phase is from 0.2 μm to 3 000 μm, preferably from 20 μm to 2 500 μm, in particular from 200 μm to 2 000 μm, and most preferably from 500 μm to 1 500 μm. In particular, in a dispersion of the invention:
the drops have a diameter greater than or equal to 250 μm, in particular greater than or equal to 500 μm, preferably greater than or equal to 750 μm, even greater than or equal to 1 000 μm, even between 250 μm and 3 000 μm, better still between 500 μm and 2 000 μm, and in particular between 750 μm and 1 000 μm, have a volume greater than or equal to 60%, even greater than or equal to 70%, preferably greater than or equal to 80%, and better still greater than or equal to 90% of the total volume of the dispersed phase and/or
at least 60%, even at least 70%, preferably at least 80%, and better still at least 90% of the drops have a mean diameter greater than or equal to 250 μm, in particular greater than or equal to 500 μm, preferably greater than or equal to 750 μm, even greater than or equal to 1 000 μm, even between 250 μm and 3 000 μm, better still between 500 μm and 2 000 μm, and in particular between 750 μm and 1 000 μm.

Determination of the volume of the drops having a particular diameter relative to the total volume of the dispersed phase lies within the general knowledge of persons skilled in the art, in particular having regard to the diameter measuring method described below.

Temperature and Pressure

Unless otherwise stated, in the remainder hereof it is considered that temperature is ambient temperature (e.g. T=25° C.±2° C.) and pressure is atmospheric pressure (760 mm de Hg, i.e. 1.013×10⁵ Pa or 1013 mbar).

Viscosity

The viscosity of a dispersion of the invention or at least of one of the phases thereof can vary extensively, which allows the obtaining of varied textures. Viscosity is measured at ambient temperature and ambient pressure following the method described in WO2017046305.

In one embodiment, a dispersion of the invention has viscosity of 1 mPa·s to 500 000 mPa·s, preferably of 10 mPa·s to 300 000 mPa·s, better still of 400 mPa·s to 100 000 mPa·s, and more particularly of 1 000 mPa·s to 30 000 mPa·s, as measured at 25° C.

Aqueous Phase

A dispersion of the invention comprises at least one aqueous phase.

The aqueous phase can correspond to the continuous phase or the dispersed phase of the dispersion of the invention, preferably the aqueous phase is the continuous phase of a dispersion of the invention.

When the continuous phase of a composition of the invention is an aqueous phase, this latter phase is preferably in the form of a gel, in particular a gel having viscosity adapted to suspend the drops of the dispersed phase and thereby contribute towards the kinetic stability and the attractive appearance of a composition of the invention. Advantageously, the continuous aqueous phase is also sufficiently shear-thinning so that when dispensed it entrains the drops together with it.

Advantageously, in particular when it is the continuous phase, the aqueous phase is not solid at ambient temperature and ambient pressure i.e. it is able to flow under its own weight.

In one embodiment, the aqueous phase has viscosity of between 400 mPa·s and 100 000 mPa·s, preferably between 800 mPa·s and 30 000 mPa·s, as measured at 25° C.

This viscosity is measured following the method described above.

The aqueous phase, preferably the continuous aqueous phase of the dispersions at least comprises water.

In addition to distilled water or deionized water, a water suitable for the invention can also be a natural spring water or floral water.

In one embodiment, the weight percentage of water in the aqueous phase, preferably the continuous aqueous phase, is at least 30%, preferably at least 40%, in particular at least 50%, and better still at least 60%, in particular between 70% and 98%, and preferably between 75% and 95%, relative to the total weight of said aqueous phase, preferably continuous aqueous phase.

The aqueous phase, in particular when it is the continuous phase of a dispersion of the invention, may also comprise at least one base. It may comprise a single base or a mixture of several different bases. The presence of at least one base in said continuous aqueous phase notably contributes towards increasing the viscosity thereof.

In the remainder of the description, this base can also be termed a «viscosity-increasing solution» or solution (BF). In one embodiment, the base contained in the aqueous phase is a mineral base.

In one embodiment, the mineral base is selected from the group composed of alkali metal hydroxides and alkaline-earth metal hydroxides.

Preferably, the mineral base is an alkali metal hydroxide and in particular NaOH.

In one embodiment, the base contained in the aqueous phase is an organic base. Among organic bases, mention can be made for example of ammonia, pyridine, triethanolamine, aminomethyl propanol, or triethylamine.

A dispersion of the invention may comprise from 0.01% to 10% by weight, preferably from 0.01% to 5% by weight, and more preferably from 0.02% to 1% by weight of base(s), preferably mineral base(s) and in particular NaOH, relative to the total weight of the aqueous phase comprising the same, and even of said dispersion.

Fatty Phase

A dispersion of the invention comprises at least one fatty phase, also termed «oily phase» in the remainder of the description.

The fatty phase can represent the continuous phase or the dispersed phase of a dispersion of the invention. Preferably, the fatty phase represents the dispersed phase of a dispersion of the invention.

Preferably, a fatty phase of a dispersion of the invention has a melting point of between 50° C. and 100° C., preferably between 60° C. and 90° C.

The melting point of a fatty phase can be measured using a differential scanning calorimeter (DSC), for example the calorimeter sold under the trade name “DSC Q2000” by TA Instruments. The protocols for sample preparation and measurement are the following: a 5 mg sample of the sample to be tested, previously heated to 80° C. and sampled under magnetic stirring using a spatula that is also heated, is placed in a sealed aluminium capsule or pan. Two tests are performed to ensure reproducibility of results. Measurements are performed on the above-mentioned calorimeter. The furnace is flushed with nitrogen. Cooling is ensured by the RCS 90 heat exchanger. The sample is subjected to the following protocol after first being brought to 20° C., it is then subjected to a first temperature rise ranging from 20° C. to 130° C., at a heating rate of 5° C./minute, then cooled from 130° C. to −80° C. at a cooling rate of 5° C./minute, and finally subjected to a second temperature rise ranging from −80° C. to 130° C. at a heating rate of 5° C./minute. During the second temperature rise, the variation is measured in the difference between the energy absorbed by the empty pan and the pan containing the sample, as a function of temperature. The melting point of the compound is the value of the temperature corresponding to the tip of the peak of the curve representing the variation in difference between absorbed energy as a function of temperature. The end-melt temperature corresponds to the temperature at which 95% of the sample has melted.

Oil(s)

Advantageously, the fatty phase may comprise at least one oil.

By «oil» it is meant a fat liquid at ambient temperature and atmospheric pressure.

As oils of the invention, mention can be made for example of:
hydrocarbon oils of vegetable origin, such as described below;
hydrocarbon oils of animal origin, such as perhydrosqualene and squalane;
synthetic esters and ethers, in particular of fatty acids such as oils having the formulas R₁COOR₂ and R₁OR₂ where R, represents the remainder of a C₈ to C₂₉ fatty acid, and R₂ is a C₃ to C₃₀ hydrocarbon chain whether or not branched, such as PurCellin oil, isononyl isononanoate, isodecyl neopentanoate, isostearyl neopentanoate, isopropyl myristate, octyldodecyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl-malate, triisocetyl citrate, heptanoates, octanoates, decanoates of fatty alcohols; polyol esters such as propylene glycol dioctanoate, neopentylglycol diheptanoate, and diethyleneglycol diisononanoate; and the esters of pentaerythritol such as pentaerythrityl tetrabehenate (DUB PTB) or pentaerythrityl tetraisostearate (Prisorine 3631);
linear or branched hydrocarbons of mineral or synthetic origin, such as paraffin oils whether or not volatile and derivatives thereof, vaseline, polydecenes, hydrogenated polyisobutene such as Parleam oil;
fatty alcohols having 8 to 26 carbon atoms such as cetyl alcohol, stearyl alcohol and the mixture thereof (cetylstearyl alcohol), or octyldodecanol;
and mixtures thereof.

Preferably, the fatty phase of a dispersion of the invention comprises at least one vegetable oil.

As hydrocarbon oil(s) of vegetable origin, mention can be made of the triglycerides of caprylic and capric acids, the triglycerides of caprylic, capric acids (also known as “MCT oil»), of myristic and stearic acids (INCI name: Caprylic/capric/myristic/stearic Triglyceride), triethylhexanoine, Limnanthes Alba seed oil (INCI name: Limnanthes Alba (Meadowfoam) Seed Oil), macadamia nut oil (INCI name: Macadamia Ternifolia Seed Oil), Rosa Canina sweet briar oil (INCI name: Rosa Canina Fruit Oil), soybean oil (INCI name: Glycine Soja (Soybean) Oil), sunflower seed oil (INCI name: Helianthus Annuus (Sunflower) Seed Oil), tribehenin (INCI name: tribehenin), triisostearin (INCI name: triisostearin), apricot kernel oil (INCI name: Prunus Armeniaca (Apricot) Kernel Oil), rice bran oil (INCI name: Oryza Sativa (Rice) Bran Oil), argan oil (INCI name: Argania Spinosa Kernel Oil), avocado oil (INCI name: Persea Gratissima Oil), evening primrose oil (INCI name: Oenothera Biennis Oil), rice germ oil (INCI name: Oryza Sativa Germ Oil), hydrogenated coconut oil (INCI name: Hydrogenated Coconut Oil), sweet almond oil (INCI name: Prunus Amygdalus Dulcis Oil), sesame seed oil (INCI name: Sesamum Indicum Seed Oil), hydrogenated rapeseed oil (INCI name: Hydrogenated Rapeseed Oil), safflower seed oil (INCI name: Carthamus Tinctorius Seed Oil), Queensland nut oil Macadamia integrifolia (INCI name: Macadamia Integrifolia Seed Oil), tricaprylin (or triacylglycerol), wheat germ oil (INCI name: Triticum Vulgare Germ Oil), borage seed oil (INCI name: Borago Officinalis Seed Oil), shea oil (INCI name: Butyrospermum Parkii Oil), hydrogenated castor oil (INCI name: Hydrogenated Castor Oil), Chinese cabbage seed oil (INCI name: Brassica Campestris Seed Oil), camellia oil and in particular Japanese camellia seed oil (INCI name: Camellia Japonica Seed Oil), green tea seed oil (INCI name: Camellia Sinensis Seed Oil), sea buckthorn oil (INCI name: Hippophae Rhamnoides Oil), Camellia Kissi seed oil (INCI name: Camellia Kissi Seed Oil), Moringa seed oil (INCI name: Moringa Pterygosperma Seed Oil), canola oil (INCI name: Canola Oil), tea seed oil (INCI name: Camellia Oleifera Seed Oil), carrot seed oil (INCI name: Daucus Carota Sativa Seed Oil), triheptanoin (INCI name: Triheptanoin), vanilla oil (INCI name: Vanilla Planifolia Fruit Oil), the glycerides of canola oil and phytosterols (INCI name: Phytosteryl Canola Glycerides), blackcurrant seed oil (INCI name: Ribes Nigrum (Black Currant) Seed Oil), karanja seed oil (INCI name: Pongamia Glabra Seed Oil), roucou oil (INCI name: Roucou (Bixa orellana) Oil), and mixtures thereof.

Advantageously, the fatty phase comprises at least one oil having a refractive index close to that of the aqueous phase, namely an oil having a refractive index at ambient temperature and atmospheric pressure preferably of between 1.2 and 1.6, more preferably between 1.25 and 1.5, in particular between 1.3 and 1.4. This embodiment is advantageous in that it allows improved transparency of the fatty phase, and hence the transparency of the dispersion of the invention. Transparency can be qualified following the method described in WO2018/167309.

Advantageously, the fatty phase of a dispersion of the invention comprises at least one, even at least two oils preferably selected from among hydrocarbon oil(s) of vegetable origin and preferably selected from among Limnanthes Alba seed oil (INCI name: Limnanthes Alba (Meadowfoam) Seed Oil), the triglycerides of caprylic, capric acids, and mixture thereof.

Preferably, a dispersion of the invention has a content of less than 1% and better still less than 0.5%, and even does not comprise a silicone oil or fluorinated oil.

A dispersion of the invention can comprise between 10% and 99.5%, preferably between 20% and 90%, better still between 30% and 85%, and in particular between 50% and 80% by weight of oil(s) relative to the total weight of the fatty phase comprising the same.

A dispersion of the invention may comprise from 1% to 50%, preferably from 5% to 40%, and better still from 10% to 25% by weight of oil(s) relative the total weight of said dispersion.

Against all expectations, the inventors have observed that a dispersion of the invention remains satisfactory in terms of kinetic stability even in the presence of high percentages of dispersed phase, in particular when the dispersion is a direct emulsion. Therefore a dispersion of the invention can advantageously comprise from 1% to 60%, in particular from 5% to 50%, preferably from 10% to 40%, and better still from 15% to 30% by weight of dispersed phase, preferably of dispersed fatty phase, or dispersed aqueous phase relative to the total weight of the dispersion.

Shell of the Drops

As mentioned above, the drops of the dispersed phase comprise a shell formed of at least one anionic polymer and at least one cationic polymer.

In other words, the drops of the invention are surrounded by a shell formed of at least one anionic polymer and at least one cationic polymer.

In the invention, the drops obtained can have a very thin shell, in particular having a thickness of less than 1% of the diameter of the drops.

The thickness of the shell is therefore preferably less than 1 μm and is therefore too small to be measured using optical methods.

In one embodiment, the thickness of the shell of the drops is less than 1 000 nm, in particular it is 1 to 500 nm, preferably less than 100 nm, advantageously less than 50 nm, most preferably less than 10 nm.

Measurement of the thickness of the shell of the drops of the invention can be performed by Small-Angle X-ray Scattering, such as implemented in CALA2

For this purpose, the drops are produced using deuterated water and are then washed three times with deuterated oil e.g. deuterated oil of hydrocarbon type (octane, dodecane, hexadecane).

After washing, the drops are transferred to the Neutron cell to determine the spectrum I(q); q being the wave vector.

From this spectrum, conventional analytical processing (REF) is applied to determine the thickness of the hydrogenated shell (non-deuterated).

In one embodiment, the shell surrounding the drops of the dispersed phase is rigidified, imparting good strength to the drops and reducing and even preventing coalescence thereof.

This shell is typically formed by coacervation i.e. by precipitation of polymers having opposite charges. Within a coacervate, the bonds linking the polymers charged together are of ionic type and are generally stronger than the bonds contained within a membrane of surfactant type.

The shell is formed by coacervation of at least two polymers having opposite polarity charges (or polyelectrolyte) and preferably in the presence of a first polymer of cationic type and a second polymer differing from the first polymer and of anionic type. Both these polymers act as rigidifying agents of the membrane.

The formation of the coacervate between these two polymers can be caused by modifying the conditions of the reaction medium (temperature, pH, concentration of reactants, etc.). The coacervation reaction results from neutralization of these two polymers having opposite polarity charges, and allows the formation of a membrane structure via electrostatic interactions between the anionic polymer and the cationic polymer. The membrane thus formed around each drop typically forms a shell which fully encapsulates the core of the drop, thereby insulating the core of the drop from the continuous phase.

Cationic Polymer

In the invention, the drops and in particular the shell of said drops comprise a polymer of cationic type. They may also comprise several polymers of cationic type. This cationic polymer is the one mentioned above which forms the shell via coacervation with the anionic polymer.

In the present application, and unless otherwise stated, by “polymer of cationic type” or «cationic polymer», it is meant a polymer comprising chemical functions of cationic type. The term cationic polyelectrolyte can also be used.

Preferably, the cationic polymer is a lipophilic polymer able to be ionized in contact with the aqueous phase.

In the invention, the cationic polymer meets the above-mentioned formula (I).

In the present invention, an alkylene radical is a divalent radical corresponding to a linear or branched alkyl group having one less hydrogen atom, able to be represented by —CₓH₂ₓ—, x being the number of carbon atoms.

In the present invention, by alkyl group it is meant a saturated, linear or branched aliphatic hydrocarbon group having x to y carbon atoms. As examples, mention can be made of methyl, ethyl, n-propyl, isopropyl, butyl, isobutyl, tertbutyl or pentyl groups.

In the present invention, a cycloalkylene radical is a divalent radical corresponding to a cycloalkyl group having one less hydrogen atom.

A cycloalkyl group according to the present invention is a cyclic carbon group having 3 to 10 carbon atoms unless otherwise stated. As examples, mention can be made of the cyclopropyl, cyclobutyl, cyclopentyl, cyclohexyl groups, etc.

Preferably, in formula (I) above, n is 1 or 2.

The compounds having n=1 will also be called trimers and the compounds having n=2 will be called pentamers.

In one embodiment, in above-mentioned formula (I), at least one among groups R⁴ and R⁵ is NH₂.

In one embodiment, in formula (I), X¹ and X² are the same and are each independently —O—, —CH₂— or —NH—.

In one embodiment, in formula (I), at least one of groups X¹ and X² is —NH—.

In one embodiment, in formula (I), X¹ and X² are —NH—.

In one embodiment, in formula (I), R⁴ and R⁵ are NH₂.

In one embodiment, in formula (I), R¹ is a linear or branched, divalent alkylene radical having 20 to 50 carbon atoms, preferably 25 to 45 carbon atoms, better still 30 to 40 carbons atoms, and most preferably 34 to 36 carbon atoms. In one embodiment, in formula (I), R¹ is a linear or branched, divalent alkylene radical having 34 to 36 carbon atoms.

In one embodiment, in formula (I), R² and R³, the same or different, preferably the same, are each independently a linear or branched, divalent alkylene radical having 20 to 50 carbon atoms, preferably 25 to 45 carbon atoms, better still 30 to 40 carbon atoms, and most preferably 34 to 36 carbon atoms. In one embodiment, in formula (I), R² and R³, the same or different, preferably the same, are each independently a linear or branched, divalent alkylene radical having 34 to 36 carbon atoms.

In one embodiment, the cationic polymer has the following formula (II):

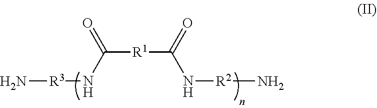

where n, R¹, R² and R³ are such as defined above.

The formula (II) compounds correspond to formula (I) compounds in which X¹═X²═NH and R⁴═R⁵═NH₂.

Preferably, in formula (II), R² and R³ are the same.

In one embodiment, in formula (II), n is 1 or 2.

In one embodiment, the cationic polymer has the following formula (III):

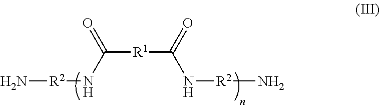

where n, R¹ and R² are such as defined above.

The formula (III) compounds correspond to formula (I) compounds in which X¹═X²═NH, R⁴═R⁵═NH₂ and R² and R³ are the same.

Preferably, in formula (III), R² is a linear or branched, divalent alkylene radical having 34 to 36 carbon atoms.

Preferably, a dispersion of the invention comprises at least one compound having the formula such as defined below:

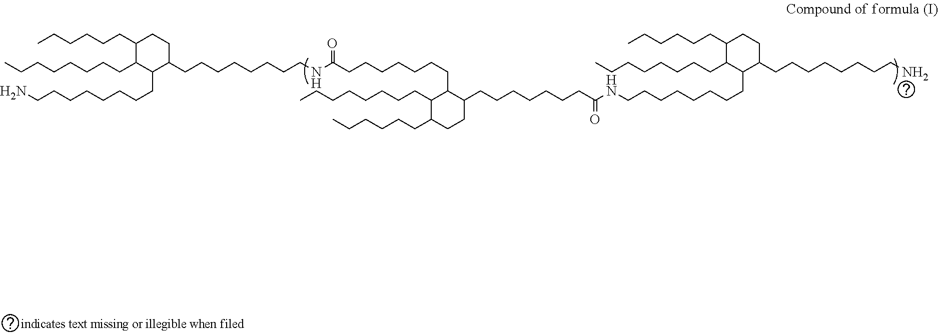

A dispersion of the invention may comprise compounds of formula (I) which differ in that:
the groups R² and R³ differ within one same compound of formula (I); and/or
the groups R¹, R², R³, R⁴ and/or R⁵ differ between two compounds of formula (I).

The inventors have observed that it may be necessary to reduce the content of cationic polymer(s) of the invention compared with the contents generally considered for amodimethicone, otherwise a wrinkled shell and/or non-spherical drops could be formed which would be cosmetically detrimental and detrimental to the kinetic stability of the dispersion. Persons skilled in the art are able to adjust the content of cationic polymer(s) to obtain a stable dispersion of the invention, and in particular comprising a non-wrinkled shell and drops that are substantially spherical.

In one embodiment, the dispersion comprises from 0.001% to 10%, preferably from 0.005% to 5%, better still from 0.01% to 2.5%, more particularly from 0.05% to 1%, even from 0.1% to 0.5% by weight of cationic polymer(s) relative to the total weight of the phase comprising the same, and in particular the fatty phase.

Anionic Polymer

In the invention, the drops and in particular the shell of said drops comprise a polymer of anionic type. They may also comprise several polymers of anionic type.

This anionic polymer is the one mentioned above which forms the shell via coacervation with the cationic polymer.

Preferably, the anionic polymer is a hydrophilic polymer able to be ionized.

By “chemical function of anionic type”, it is meant a chemical function AH capable of yielding a proton to give a function A⁻. Depending on the conditions of the medium in which it is included, the polymer of anionic type therefore comprises chemical functions in AH form, or else in the form of its conjugate base A⁻.

As examples of chemical functions of anionic type, mention can be made of the carboxylic acid functions-COOH, optionally present in carboxylate anion form —COO⁻.

As examples of polymers of anionic type, mention can be made of any polymer formed by polymerization of monomers of which at least one part carries chemical functions of anionic type, such as carboxylic acid functions. Said monomers are for example acrylic acid, maleic acid, or any ethylenically unsaturated monomer comprising at least one carboxylic acid function. For example, it may be an anionic polymer comprising monomer units comprising at least one chemical function of carboxylic acid type.

Preferably, the anionic polymer is hydrophilic i.e. soluble or dispersible in water.

Among the examples of polymers of anionic type suitable for implementing the invention, mention can be made of the copolymers of acrylic acid or maleic acid with other monomers such as acrylamide, alkyl acrylates, C₅-C₃ alkyl acrylates, C₁₀-C₃₀ alkyl acrylates, C₁₂-C₂₂ alkyl methacrylates, methoxypolyethyleneglycol methacrylates, hydroxyester acrylates, crosspolymer acrylates, and mixtures thereof. In the invention, a polymer of anionic type is preferably a carbomer such as described below. This polymer can also be a crosslinked acrylates/C₁₀₋₃₀ alkyl acrylate copolymer (INCI name: acrylates/C₁₀₋₃₀ alkyl acrylate Crosspolymer).

In one embodiment, the shell of the drops comprises at least one anionic polymer such as a carbomer.

In one embodiment, the anionic polymer is a polymer comprising monomer units comprising at least one carboxylic acid chemical function, preferably selected from among carbomers or an acrylates/C₁₀₋₃₀ alkyl acrylate crosspolymer, and preferably a carbomer.

In the invention, and unless otherwise stated, by “carbomer” it is meant an optionally crosslinked homopolymer, derived from polymerization of acrylic acid. It is therefore a poly(acrylic acid), optionally crosslinked. Among the carbomers of the invention, mention can be made of those marketed under the trade names Tego® Carbomer 340FD by Evonik, Carbopol® 981 by Lubrizol, Carbopol® ETD 2050 by Lubrizol, or Carbopol® Ultrez 10 by Lubrizol.

In one embodiment, by “carbomer” or “Carbopol®», it is meant an acrylic acid polymer of high molecular weight crosslinked with allyl sucrose, or allyl pentaerythritol ethers (Handbook of Pharmaceutical Excipients, 5^{th} Edition, pIII). For example, it is Carbopol®910, Carbopol®934, Carbopol®934P, Carbopol®940, Carbopol®941, Carbopol®71G, Carbopol®980, Carbopol®971P or Carbopol®974P. In one embodiment, the viscosity of said carbomer is between 4 000 and 60 000 cP at 0.5% w/w.

Carbomers have other names: polyacrylic acids, carboxyvinyl polymers or carboxy polyethylenes.

A dispersion of the invention may comprise from 0.01% to 10%, preferably from 0.05% to 5%, and more preferably from 0.10% to 2.5%, even from 0.5% to 1% by weight of anionic polymer(s), in particular of carbomer(s), relative to the total weight of said dispersion.

In the invention, the dispersions of the invention may comprise a carbomer and an acrylates/C₁₀₋₃₀ alkyl acrylate crosspolymer.

The aqueous phase of the invention may also comprise a least one crosslinked polymer or at least one crosslinked copolymer, said crosslinked polymer or crosslinked copolymer comprising at least one unit derived from polymerization of one of the following monomers: acrylic or methacrylic acid, alkyl acrylate or methacrylate having 1 to 30 carbon atoms, or the salts thereof.

This is notably the case when a dispersion of the invention comprises at least one fragrance ingredient such as defined below.

The aqueous phase may also comprise a mixture of crosslinked polymers or a mixture of crosslinked copolymers, or a mixture of crosslinked polymer(s) and crosslinked copolymer(s).

In the invention, the term “unit derived from polymerization of a monomer” it is meant that the polymer or copolymer is a polymer or copolymer obtained by polymerization of said monomer.

In one embodiment, the crosslinked polymer or crosslinked copolymer is a crosslinked polyacrylate.

The crosslinked copolymers and polymers of the invention are anionic.

In one embodiment, the copolymer is a copolymer of unsaturated carboxylic acid and an unsaturated carboxylate of C₁₋₃₀ alkyl, preferably C₁-C₄. Said copolymer comprises at least one hydrophilic repeat unit of unsaturated olefin carboxylic acid type and at least one hydrophobic repeat unit of the type (C₁-C₃₀) alkyl ester of unsaturated carboxylic acid.

Preferably, these copolymers are selected from among those having a hydrophilic repeat unit of unsaturated olefin carboxylic acid type which corresponds to the monomer of following formula (IV):

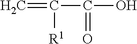

where: R₁ is H or CH₃ or C₂H₅, i.e. acrylic acid, methacrylic acid or ethacrylic acid repeat units, and in which the hydrophobic repeat unit of (C₁-C₃₀) alkyl ester of unsaturated carboxylic acid corresponds to the monomer of following formula (V):

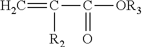

where: R₂ is H or CH₃ or C₂H₅ (i.e. acrylate, methacrylate or ethacrylate repeat units) and preferably H (acrylate repeat units) or CH₃ (methacrylate repeat units), R₃ designating a C₁-C₃₀ alkyl radical, preferably a C₁-C₄ alkyl radical.

Among this type of copolymers, more particular use is made of those formed from a mixture of monomers comprising:
(i) essentially acrylic acid,
(ii) an ester of formula (IV) described above and in which R₂ is H or CHs, R₃ designating an alkyl radical having 1 to 4 carbon atoms.
(iii) and a crosslinking agent which is a well-known co-polymerizable unsaturated polyethylene monomer such as diallyl phthalate, trimethylolpropane tri(meth)acrylate, diallyl itaconate, diallyl fumarate, diallyl maleate, zinc (meth)acrylate, allyl (meth)acrylate, divinylbenzene, (poly)ethyleneglycol dimethacrylate, methylene-bis-acrylamide, and castor oil.

In one embodiment, the crosslinked polymer or crosslinked copolymer is a polymer of copolymer of acrylic acid and/or methacrylic acid, and/or alkyl acrylate having 1 to 30 carbon atoms, preferably 1 to 4 carbon atoms, and/or alkyl methacrylate having 1 to 30 carbon atoms, preferably 1 to 4 carbon atoms.

In one embodiment, the crosslinked copolymer is a crosslinked copolymer of methacrylic acid and alkyl acrylate having 1 to 4 carbon atoms, preferably 2 carbon atoms.

In the invention, and unless otherwise stated, by crosslinked copolymer of methacrylic acid and alkyl acrylate having 1 to 4 carbon atoms», it is meant a crosslinked copolymer resulting from polymerization of a methacrylic acid monomer and an alkyl acrylate monomer having 1 to 4 carbon atoms.

Preferably, in this copolymer, the methacrylic acid represents from 20% to 80% by weight, preferably from 35% to 65% by weight of the total weight of the copolymer.

Preferably, in this copolymer, the alkyl acrylate represents from 15% to 80% by weight, preferably from 35% to 65% by weight of the total weight of the copolymer.

In particular, the alkyl acrylate is selected from among alkyl methacrylate, ethyl acrylate and butyl acrylate.

In one embodiment, the crosslinked polymer or crosslinked copolymer of the invention present in the continuous aqueous phase is selected from the group formed by the following polymers or copolymers: Acrylates Copolymer, Acrylates crosspolymer-4, Acrylates crosspolymer-3, Polyacrylate-2 Crosspolymer and Polyacrylate-14 (INCI names).

Among said above polymers, in the present invention particular preference is given to the products sold by LUBRIZOL under the trade names Fixate Superhold (INCI name=Polyacrylate-2 Crosspolymer), Fixate Freestyle Polymer (INCI name=Acrylates crosspolymer-3), Carbopol® Aqua SF1 (INCI name=Acrylates copolymer) and Carbopol® Aqua SF2 (INCI name=Acrylates crosspolymer-4).

Preferably, the crosslinked copolymer is Carbopol® Aqua SF1 (INCI name=Acrylates copolymer).

In one embodiment, the crosslinked copolymer is selected from among the crosslinked copolymers of acrylic or methacrylic acid and alkyl acrylates having 1 to 4 carbon atoms.

In the invention, the dispersion of the invention may comprise from 0.1% to 10% by weight, preferably from 0.5% to 8% by weight, and most preferably from 1% to 3% by weight of crosslinked polymer(s) or crosslinked copolymer(s) relative to the total weight of said dispersion.

In the invention, the dispersions of the invention may comprise a carbomer and a crosslinked copolymer Carbopol® Aqua SF1 (INCI name=Acrylates copolymer).

In one embodiment, the cationic polymer is a lipophilic polymer able to be ionized in contact with an aqueous phase, and the anionic polymer is a hydrophilic polymer able to be ionized.

In one embodiment, the drops of the dispersion of the invention comprise a core that is liquid, or at least partially gelled or at least partially thixotropic, said core being single-phase or comprising an intermediate drop of an intermediate phase, and at least one, preferably only one, inner drop of an inner phase arranged in the intermediate drop, the intermediate phase and the inner phase being non-miscible with each other at ambient temperature and atmospheric pressure.

The intermediate phase is preferably based on a solution non-miscible with the continuous phase, and the intermediate phase is preferably based on solution non-miscible with the inner phase at ambient temperature and atmospheric pressure.

Therefore the solubility of the intermediate phase with the continuous phase and inner phase is advantageously less than 5% by weight, and conversely, in which case:
when the continuous phase is aqueous, the intermediate phase is oily and the inner phase is aqueous;
when the continuous phase is oily, the intermediate phase is aqueous and the inner phase is oily; and
when the continuous phase is aqueous, the intermediate phase is oily and the inner phase is oily.

Advantageously, the intermediate phase comprises at least one gelling agent (or texturizing agent), in particular such as defined below. The gelling agent particularly contributes to improving the suspension of the inner drop(s) arranged in the intermediate drop, and prevents/avoids phenomena of creaming or sedimentation of the inner drop(s) arranged in the intermediate drop.

Lipophilic Gelling Agent

In one embodiment, the fatty phase also comprises at least one lipophilic gelling agent.

Said gelling agent differs from the anionic and cationic polymers and oils described above.

In the invention, and unless otherwise stated, by «gelling agent» it is meant an agent allowing an increase in the viscosity of the oily phase of the dispersion devoid of said gelling agent, for example allowing a final viscosity to be reached of the gelled fatty phase higher than 20 000 mPa·s, preferably higher than 50 000 mPa·s, better still higher than 100 000 mPa·s, and most particularly higher than 200 000 mPa·s.

Preferably, the lipophilic gelling agent of the invention is selected from among organic or mineral, polymeric or molecular lipophilic gelling agents; the fats solid at ambient temperature and pressure particularly being selected from among waxes, pasty fats, butters, and their mixtures; and mixtures thereof.

These lipophilic gelling agents are described in more detail in WO2021234135.

In one preferred embodiment, the lipophilic gelling agent is selected from the group composed of polymeric lipophilic gelling gents, and in particular selected from the group composed of polyacrylates, esters of dextrin and fatty acid(s), esters of glycerol and fatty acid(s), polyamides, and mixtures thereof.

Particular mention can be made of the esters of dextrin and fatty acid(s) marketed under the trade names Rheopearl® KL2 (INCI name: dextrin palmitate), Rheopearl® TT2 (INCI name: dextrin palmitate ethylhexanoate), and Rheopearl® MKL2 (INCI name: dextrin myristate) by Miyoshi Europe.

In one particularly preferred embodiment, the lipophilic gelling agent is selected from among Castor Oil/IPDI Copolymer (and) Caprylic/Capric Triglyceride, particularly marketed under the trade name Estogel® M by PolymerExpert, Hydrogenated Castor Oil/Sebacic Acid Copolymer and the derivatives thereof, respectively marketed in particular under the trade names Estogel® Green (or Estogel® G) and Estogel® Green 40 by PolymerExpert, Caprylic/Capric Triglyceride (and) Polyurethane-79 marketed in particular under the trade name OILKEMIA™ 5S polymer by Lubrizol, Trihydroxystearin marketed in particular under the trade name THIXCIN® R by Elementis Specialties, and mixtures thereof, and better still Castor Oil/IPDI Copolymer (and) Caprylic/Capric Triglyceride.

In one particular embodiment, a dispersion of the invention, in particular the fatty phase, does not comprise an elastomer gel comprising at least one dimethicone, in particular such as marketed by NuSil Technology under the trade name CareSil™ CXG-1104 (INCI: Dimethicone (and) Dimethicone/Vinyl Dimethicone Crosspolymer).

Advantageously, a lipophilic gelling agent is a heat-sensitive gelling agent, namely which reacts to heat, and in particular it is a gelling agent solid at ambient temperature and liquid at a temperature higher than 50° C., preferably higher than 60° C., and better still higher than 70° C. Preferably, a heat-sensitive lipophilic gelling agent of the invention has a melting point of between 50° C. and 130° C., and preferably between 60° C. and 120° C.

Advantageously, a fatty phase gelling agent of the invention is a thixotropic gelling agent or able to impart thixotropic behaviour to the solution in which it is contained. Said thixotropic gelling agent is particularly selected from among fumed silicas optionally with hydrophobic treatment, described previously.

Persons skilled in the art will ensure that the lipophilic gelling agent(s) and/or the quantity thereof are chosen in a manner to meet the restrictions imposed by the production method used (in particular of «non-microfluidic» or «microfluidic type») to produce the dispersion of the invention. These adjustments lie within the competence of skilled persons having regard to the teaching of the present description.

In one embodiment, a dispersion of the invention may comprise from 0.5% to 70%, preferably from 1% to 60%, in particular from 1.5% to 50%, more preferably from 2% to 40%, further preferably from 5% to 30%, and most preferably from 10% to 20% by weight of lipophilic gelling agent(s) relative to the total weight of the fatty phase in which they are contained.

Additional Compound(s)

A dispersion of the invention, in particular the aqueous phase and/or the fatty phase may further comprise at least one additional compound differing from the above-mentioned cationic and anionic polymers, lipophilic gelling agents and oils.

The dispersions of the invention can therefore further comprise powders; flakes; colouring agents selected in particular from among water-soluble or non-water soluble, lipophilic or non-lipophilic, organic or inorganic colouring agents; materials with optical effect, liquid crystals, and mixtures thereof; particulate agents insoluble in the fatty phase; fillers of organic or mineral type; preserving agents; moisturizers; stabilizers; chelating agents; emollients; modifying agents selected from among texturizing agents (or gelling agents), modifiers of pH, of osmotic strength and/or modifiers of refractive index etc. . . . or any usual cosmetic additive; and mixtures thereof.

In one embodiment, the particulate agents insoluble in the fatty phase of the drops are selected from the group composed of pigments, nacre, ceramics, polymers and in particular acrylic polymers, and mixtures thereof.

The dispersions of the invention may further comprise at least one biological/cosmetic ingredient selected from among hydrating agents, healing agents, depigmenting agents, UV filters, peeling agents, antioxidants, active substances stimulating the synthesis of dermal and/or epidermal macromolecules, dermo-relaxants anti-perspiration agents, soothing agents and/or anti-ageing agents, and mixtures thereof.

Hydrophilic Texturizing Agent(s)

An aqueous phase of the invention may further comprise at least one hydrophilic texturizing agent differing from the above-mentioned cationic and anionic polymers, oils, lipophilic gelling agents and base.

Said hydrophilic texturizing agent advantageously allows modulation of the viscosity and sensory perception of a dispersion of the invention, and even allows improved kinetic stability. When included in a continuous aqueous phase, the hydrophilic texturizing agent may impact the suspensibility of the drops.

As hydrophilic texturizing agents i.e. water-soluble or water-dispersible, and therefore able to be contained in the aqueous phase of a composition of the invention, mention can be made of:
natural texturizing agents, particularly selected from among algae extracts, plant exudates, seed extracts, microorganism exudates such as alcasealan (INCI name: Alcaligenes Polysaccharides), and other natural agents, in particular hyaluronic acid;
semi-synthetic texturizing agents, selected in particular from among cellulose derivatives and modified starches;
synthetic texturizing agents selected in particular from among the homopolymers of (meth)acrylic acid or one of the esters thereof, the copolymers of (meth)acrylic acid or one of the esters thereof, AMPS copolymers (2-acrylamido-2-methylpropane sulfonic acid), associative polymers,
other texturizing agents selected in particular from among polyethylene glycols (marketed under the trade name Carbowax®), clays, silicas such as those marketed under the trade names Aerosil® 90/130/150/200/300/380), glycerin, and
mixtures thereof.

By «associative polymer» in the meaning of the present invention, it is meant any amphiphilic polymer having in its structure at least one fatty chain and at least one hydrophilic portion; the associative polymers conforming to the present invention can be anionic, cationic, non-ionic, or amphoteric; in particular, they are those described in FR 2 999 921. Preferably, they are amphiphilic and anionic associative polymers, and amphiphilic and non-ionic associative polymers such as described below.

These hydrophilic texturizing agents are described in more detail in FR3041251.

Evidently, those skilled in the art will ensure that any additional compound(s) and/or the quantity thereof are chosen so that the advantageous properties of the dispersion of the invention are not or are not substantially deteriorated by the envisaged addition. In particular, the type and/or quantity of said additional compound(s) are dependent on the aqueous or fatty nature of the phase under consideration in the dispersion of the invention. These adjustments lie within the reach of persons skilled in the art.

Preparation Method

The dispersions of the invention can be prepared with different methods.

Therefore, the dispersions of the invention have the advantage that they can be prepared with a simple «non-microfluidic» method, namely by simple emulsification.

As in a conventional emulsion, an aqueous solution and a fatty solution are prepared separately. It is the addition under agitation of the fatty phase to the aqueous phase which creates the direct emulsion.

The viscosity of the continuous aqueous phase can be controlled in particular by acting on the quantity of anionic polymer (carbomer in particular) and the pH of the solution and/or the type and/or the content of hydrophilic texturizing agent(s). In general, the pH of the aqueous phase is lower than 4.5, which can entail the addition of a third viscosity-increasing solution (BF) at a final stage to reach a pH of between 5.5 and 6.5.

The viscosity of the phases and the shear force applied to the mixture are the two main parameters which impact the size and monodispersity of the drops of the dispersion.

Those skilled in the art are able to adjust the non-microfluidic method to meet the criterion of mean diameter of the drops of the dispersion of the invention.

The dispersions of the invention can also be prepared following a microfluidic method, in particular as described in international applications WO2012/120043, WO2015/055748 or WO2019/145424.

According to this embodiment, the drops obtained with these microfluidic methods have a uniform size distribution.

Preferably, the dispersions of the invention are composed of a population of monodisperse drops, in particular such that they have a mean diameter D̅ of 500 μm to 3 000 μm and a coefficient of variation Cv lower than 10%, even lower than 3%.

In the present description, by “monodisperse drops” it is meant the fact that the population of drops of the dispersion of the invention has a uniform size distribution.

Monodisperse drops have good monodispersity. Conversely, drops having poor monodispersity are said to be “polydisperse”.

In one embodiment, the mean diameter D̅ of the drops is measured for example by analysis of a photograph of a batch composed of N drops, using image processing software (Image J). Typically, with this method, the diameter is measured in pixels then expressed in μm as a function of the size of the receptacle containing the drops of the dispersion.

Preferably, the value of N is chosen to be higher than or equal to 30, so that this analysis reflects the distribution of the diameters of the drops of said emulsion in statistically significant manner.

The diameter Di of each drop is measured, and the mean diameter D̅ is obtained by calculating the arithmetic mean of these values:

$$\overline{D} = \frac{1}{N} {\sum }_{i = 1}^{N} {D}_{i}$$

From these values Dᵢ, it is also possible to obtain the standard deviation a of the diameters of the drops of the dispersion:

$$σ = \sqrt{\frac{{\sum }_{i = 1}^{N} {( {D}_{i} - \overline{D} )}^{2}}{N}}$$

The standard deviation σ of a dispersion reflects the distribution of the diameters Dᵢ of the drops of the dispersion around the mean diameter D̅.

Knowing the mean diameter D̅ and the standard deviation σ of a dispersion, it is possible to determine that 95.4% of the population of drops is found within the range of diameters [D̅−2σ; D̅+2σ] and that 68.2% of the population is found within the range [D̅−σ; D̅+σ].

To characterize the monodispersity of the dispersion according to this embodiment of the invention, the coefficient of variation can be calculated:

$${C}_{v} = \frac{σ}{\overline{D}}$$

This parameter reflects the distribution of the diameters of the drops as a function of the mean diameter thereof.

The coefficient of variation Cv of the diameters of the drops according to this embodiment of the invention is lower than 10%, de preferably lower than 5%, even lower than 3%.

Alternatively, monodispersity can be evidenced by placing a sample of dispersion in a flask having a constant circular cross-section. Gentle agitation by rotating a quarter of a turn over one half-second about the axis of symmetry passing through the flask, followed by a rest time of one half-second is performed before repeating the operation in opposite direction, this agitation being performed four successive times.

The drops of the dispersed phase organize themselves into a crystalline form when they are monodisperse. They therefore exhibit stacking in a pattern repeating itself in the three dimensions. It is then possible to observe regular stacking which indicates good monodispersity, irregular stacking translating polydispersity of the dispersion.

To obtain monodisperse drops, it is also possible to implement the microfluidic technique (Utada et al. MRS Bulletin 32, 702-708 (2007); Cramer et al. Chem. Eng. Sci. 59, 15, 3045-3058 (2004)), and more particularly microfluidic devices of co-flow type (the fluids move in the same direction) or flow-focusing type (the fluids move in different directions and typically in opposite directions).

The presence of lipophilic gelling agent(s) and/or hydrophilic texturizing agent(s) may necessitate adjustments in the method for preparing a dispersion of the invention. In particular, the method for preparing a dispersion of the invention may comprise a heating step (between 40° C. and 150° C., in particular between 50° C. and 90° C.) of the fatty phase and/or aqueous phase before, and even also during the mixing/contacting of said fatty phase with the aqueous phase.

The present invention concerns a method for preparing a dispersion such as defined above, comprising the following steps:
optionally, heating an oily fluid F1 and/or an aqueous fluid FE, to a temperature of 40° C. to 150°;
placing the aqueous fluid FE and oily fluid F1 in contact; and
forming fatty phase drops composed of the oily fluid FI dispersed in a continuous aqueous phase composed of the aqueous fluid FE, or conversely, said drops comprising a shell insulating the core of the drops,

wherein:
the oily fluid FI comprises at least one oil and at least one cationic polymer of formula (I) such as defined above, and optionally also at least one lipophilic gelling agent; and
the aqueous fluid FE comprises at least water and at least one anionic polymer, in particular a carbomer, and optionally also at least one hydrophilic texturizing agent.

In one embodiment, the fluid FI is initially prepared by mixing a fatty phase intended to form the core of the drops, at least one oil and at least one cationic polymer such as previously defined, and optionally at least one lipophilic gelling agent and/or at least one additional compound such as mentioned above.

In one embodiment the fluid FE is initially prepared by mixing an aqueous phase intended to form the continuous phase of the dispersion with optionally at least one base, at least one anionic polymer such as previously defined, and optionally at least one additional compound, preserving agents and/or other water-soluble products such as glycerine.

In one embodiment, the continuous aqueous phase of the formed dispersion comprises and is even the aqueous phase of the fluid FE. The anionic polymer contained in said fluid FE is particularly used to form the shell of the drops. Said anionic polymer also contributes towards increasing the viscosity of the fluid FE when it is intended to represent the continuous aqueous phase.

In one embodiment, the step to form the drops may further comprise a step to inject a solution increasing the viscosity of the continuous aqueous phase of the fluid FE. Preferably, the viscosity-increasing solution is aqueous. This viscosity-increasing solution is typically injected into the external aqueous fluid FE after forming the dispersion of the invention, and hence after forming of the drops.

In one embodiment, the viscosity-increasing solution comprises a base, in particular an alkali hydroxide such as sodium hydroxide.

Advantageously, a method of the invention, after the drop formation step, further comprises a cooling step. This cooling step advantageously allows accelerated cooling kinetics of the formed dispersion, thereby preventing risks of coalescence and fragmentation of the drops after formation thereof (between 10 and 30° C.).

In particular, the cooling step is conducted at a temperature lower than the melting point of the lipophilic gelling agent(s) and/or hydrophilic texturizing agent(s) used, and preferably at a temperature lower than the lowest melting point among those of the lipophilic gelling agent(s) and/or hydrophilic texturizing agent(s) used. In particular, the cooling step is conducted at a temperature lower than ambient temperature, preferably at a temperature of 0° C. to 25° C., preferably 5° C. to 20° C., and better still at 10° C. to 15° C. This cooling step can be performed by passing the dispersion through a multitube exchanger preferably mounted in the immediate vicinity of the outlet(s) of the microfluidic device.

Uses

Preferably, a dispersion of the invention is dedicated to a topical application.

Preferably, the dispersion of the invention can be used directly after the aforementioned preparation methods as a composition and in particular a cosmetic composition. The dispersion of the invention, when prepared with a microfluidic method such as described above, can also be used as a composition, in particular a cosmetic composition, after separation of the drops and re-dispersion thereof in a second suitable phase.

The invention also concerns the use of a dispersion of the invention to prepare a composition, in particular a cosmetic composition.

The present therefore also concerns a composition, in particular a cosmetic composition, comprising at least one dispersion of the invention, optionally in association with a physiologically acceptable medium.

The dispersions or composition of the invention can notably be used in the sphere of cosmetics.

In addition to the above-mentioned ingredients, they may comprise at least one physiologically acceptable medium.

In the invention, and unless otherwise stated, by “physiologically acceptable medium”, it is meant a medium suitable for cosmetic applications, and suitable in particular for application of a composition of the invention onto keratin material, in particular the skin and/or hair, and more particularly the skin.

The physiologically acceptable medium is generally adapted to the type of support onto which the composition is to be applied, and to the desired appearance of the packaging of the composition.

In one embodiment, the physiologically acceptable medium is represented directly by the continuous aqueous phase such as described above.

The cosmetic compositions of the invention can be a cream for example, an emulsion, a lotion, serum, gel and oil for the skin (hands, face, feet, etc.), a foundation (liquid, paste), a bath and shower preparation (salts, foams, oil, gels, etc.), a hair care product (hair dyes and bleaches), a cleansing product (lotions, powders, shampoos), a hair conditioning product (lotions, creams, oils), a hair styling product (lotions, lacquers, brilliantine), a shaving product (soaps, foams, lotions, etc.), a product intended to be applied to the lips, a sunscreen product, a self-tanning product, a skin whitening product, an anti-wrinkle product. In particular, the cosmetic compositions of the invention can be an anti-age serum, a youth serum, a moisturizing serum or fragrant water.

The present invention also concerns a non-therapeutic cosmetic treatment method, in particular make-up and/or care method of keratin material, in particular the skin, lips or hair, comprising at least one step to apply to said keratin material at least one dispersion such as defined above or at least one composition such as defined above.

### EXAMPLES

### Example 1—Synthesis of a Cationic Polymer of the Invention

This Example 1 is based on use of the raw material marketed by Croda under the trade name Priamine® 1075.

TABLE 1
Priamine ® 1075

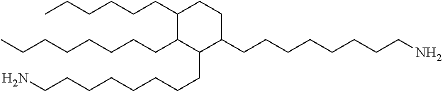 [SMILES: CCCCCCCCC1C(CCCCCC)CCC(CCCCCCCCN)C1CCCCCCCCN]

| Property | Unit | Value |
|---|---|---|
| Amine number | mgKOH/g | 205 |
| Gardner colour | | 1 |
| Dynamic viscosity | mPa.s at 25° C. | 210 |
| ISCC PLUS bio-content | % | 100 |
| certified sustainable | | |

This Example 1 is based on use of the raw material marketed by Croda under the trade name Pripol® 1009:

TABLE 2
Pripol ® 1009

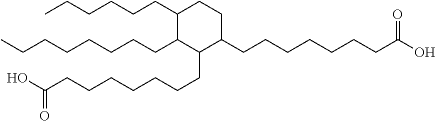 [SMILES: CCCCCCCCC1C(CCCCCC)CCC(CCCCCCCC(=O)O)C1CCCCCCCC(=O)O]

| Property | Unit | Value |
|---|---|---|
| Acid number | mgKOH/g | 196 |
| APHA colour | Hazen at 25° C. | 50 |
| lodine number | mgKOH/g | 7 |
| ISCC PLUS bio-content | % | 100 |
| certified sustainable | | |

The purification solvent used is Cetiol® C5C (INCI: Coco-Capyrylate/Caprate) marketed by BASF.

### Step 1—Synthesis:

546 g of Priamine® 1075 and 142 g de Pripol® 1009 were placed in a 1 L reactor. The reaction mixture was left under mechanical agitation at 140° C. for 23 h in a vacuum. The reaction was monitored by infra-red, in particular by growth and stabilization of the peak related to the amide functions (CONH, 1646 cm⁻¹). Size exclusion chromatography (SEC) with polystyrene calibration indicated the presence in the reaction product of 44% «Priamine» (Mn theoretical 546 g/mol), 35% trimers (Mn theoretical 1663 g/mol), 15% pentamers (Mn theoretical 2780 g/mol), and 6% high-order oligomers. The amine number of the reaction medium was measured by titration at 2.2 equivalents/kg.

### Step 1—Purification:

10 g of the reaction product obtained after step 1 and 90 g of Cetiol® C5C were mixed together. The mixture was centrifuged at 5000 rpm, at 15° C. for 15 minutes and placed at 4° C. overnight. The mixture was subsequently recentrifuged at 5000 rpm at 4° C., for 15 to 20 minutes. The residue containing «Priamine» (30%), trimers (37%), pentamers (20%) and high-order oligomers (13%) was removed. The percentage evaluated by gravimetric of formula (I) compound(s) in solution in the oil was about 8.2% and the composition thereof estimated by calculation was 38% «Priamine», 42% trimers, 14% pentamers and 6% high-order oligomers. The amine number: «formula (I) compounds/Cetiol® C5C» measured by titration was 0.205 eq/kg.

TABLE 3
Compound of formula (I) resulting from the reaction between Priamine ® 1075 and Pripol ® 1009

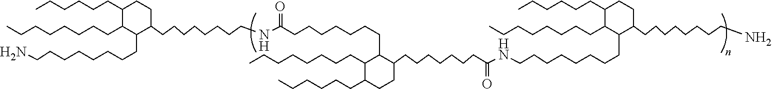

### Example 2—Skin Care Macroscopic Dispersions

For this Example, two dispersions A and B were prepared of macroscopic drops of a gelled fatty phase dispersed in a continuous aqueous phase, using a microfluidic device such as described in WO2017046305. The compositions of the (fluid) phases allowing the preparation of these two dispersions were as follows:

TABLE 4

| | | | A | B |
|---|---|---|---|---|
| | | | (Comp.) | (Invtn.) |
| Fluid | Name | INCI | % w/w | % w/w |
| IF | DUB ININ Grade A | Isononyl isononanoate | q.s .* | |
| (gelled | Meadowfoam oil | Limnanthes alba seed oil | 18.00 | |
| fatty | Rheopearl ® KL2 | Dextrin palmitate, Palmitic | 15.00 | |
| phase) | | Acid, Aqua | | |
| | PHAT BLUE DC6204 | CI 61565 (and) CI 60725 | 0.00092 | |
| | CAS-3131 PILOT | Amodimethicone | 0.20 | 0 |
| | Cationic polymer of | — | 0 | 0.041 |
| | formula (III)** | | | |
| | Total | | 100 | |
| OF | Osmosis water | Aqua | q.s .* | |
| (continuous | Microcare ® PE | Phenoxyethanol, aqua | 0.89 | |
| aqueous | Microcare ® emollient PTG | Pentylene glycol, aqua | 2.22 | |
| phase) | Glycerine codex | Glycerin, aqua | 11.11 | |
| | Edeta BD | Disodium EDTA | 0.044 | |
| | Carbopol ® ETD 2050 | Carbomer | 0.33 | |
| | polymer | | | |
| | Sodium hydroxide | Sodium hydroxide | 0.013 | |
| | pellets PRS codex | | | |
| | Total | | 100 | |
| BF | Osmosis water | Aqua | q.s.* | |
| (base) | Sodium hydroxide | Sodium hydroxide | 0.35 | |
| | pellets PRS codex | | | |

| | |
|---|---|
| Total | 100 |

*q.s.: as much as suffices
**Cationic polymer of formula (III) obtained in Example 1, and hence in which R1 is a divalent alkylene radical having 34 carbon atoms, and R2 is a divalent alkylene radical having 36 carbon atoms.

Preparation Protocol:

For OF:

The Phenoxyethanol, Pentyleneglycol and EDTA are incorporated in water. The mixture is left under agitation for 5 min.

The carbomer is dispersed in the preceding mixture under agitation for 30 minutes using a dispersion blade.

After the addition of glycerine, the mixture is left under further agitation for 10 min.

The addition is then made of sodium hydroxide and the solution is mixed for 10 minutes.

For IF:

The lipophilic cationic polymer (i.e. amodimethicone or cationic polymer of formula (III)) is added to the isononyl isononanoate and mixed with a magnetic stir bar for 5 min. The colouring agent PHAT BLUE DC6204 is afterwards added under agitation.

Under agitation, the addition is made of Meadowfoam oil.

The mixture is heated to 80° C. and Rheopearl® micr KL2 is added under magnetic stirring until a homogeneous solution is obtained.

The heated IF solution is charged into a syringe connected to a heat source to maintain the IF at a warm temperature (80° C.). To reduce heat losses, the microfluidic device is installed directly at the syringe outlet.

BF: the sodium hydroxide and water are mixed with a magnetic stir bar for 5 min.

In these tests, the flow rates per nozzle given below were used:

| | |
|---|---|
| OF | 150 mL/hr |
| IF | 20.35 mL/hr |
| BF | 16.667 mL/hr |

In both cases, dispersions were obtained comprising spherical drops having a mean diameter of 1000 μm.

The cationic polymer of formula (I) therefore does not hamper good formation of the dispersion or the robustness of the production method.

In addition, the kinetics of the reaction of the cationic polymer of formula (I) with the «carbomer», and hence the formation of a shell of coacervate, appear similar to those obtained with Amodimethicone.

The cationic polymer of formula (I) is therefore able to substitute for amodimethicone, at least for the production of dispersions and in particular dispersions having macroscopic drops.

Visual analysis was carried out of the kinetic stability of the two dispersions in terms of adhesion, aggregation and turbidity (or opacification) of the aqueous phase.

For this purpose, after production, each of the dispersions A and B was placed in three 30 ml polypropylene (PP) containers that were half filled. After 1 day at ambient temperature, each sample was subjected to one of the three shaker tests below (one container per test), namely:
roll test (i.e. circular, horizontal movement): Wheaton reference, for 1 hour;
orbital shaker (i.e. circular, vertical movement): Heidolph Unimax 1010 reference, for 1 hour; and
3D mixer (i.e. random movements): for 6 minutes.

TABLE 5

| ADHESION | AGGREGATION | TURBIDITY |
|---|---|---|
| Attaching of drops | Agglomeration of drops together | Transfer of the fatty phase |
| to the wall | (agrégation tends to promote | into the continuous aqueous |
| of packaging | coalescence) | phase |

Score Criteria:

TABLE 6

| SCORE | | | | |
|---|---|---|---|---|
| CRITERIA | 0 | 1 | 2 | 3 |
| ADHESION | No adhesion | Slight | Moderate | Strong |
| | | adhesion | adhesion | adhesion |
| AGGREGATION | No aggregation | Slight | Moderate | Strong |
| | | aggregation | aggregation | aggregation |
| GEL TURBIDITY | Transparent gel | Slightly | Moderately | Cloudy |
| | | cloudy gel | cloudy gel | gel |

Results:

TABLE 7

| | A | B |
|---|---|---|
| | (Comp.) | Invtn.) |
| ADHESION | 1 | 1 |
| AGGREGATION | 0 | 0 |
| GEL | 0 | 0 |
| TURBIDITY | | |

It is concluded from the above that the impact of a cationic polymer of the invention in terms of kinetic stability on a dispersion which, in addition, is provided with macroscopic drops, is similar to that of amodimethicone.

### Example 3—Macroscopic Dispersions of Foundation Type

For this Example, two dispersions C and D of macroscopic drops were prepared of a gelled fatty phase dispersed in a continuous aqueous phase using a microfluidic device such as described in WO2019145424. The compositions of the (fluid) phases allowing the preparation of these two dispersions were the following:

TABLE 8

| | | | C | D | |
|---|---|---|---|---|---|
| | | | (Comp.) | (Invtn.) | |
| | | | % w/W | | |
| Name | | INCI name | Phases | | |
| AQUEOUS GEL PHASE (=OF) | | | 100.00 | | A |
| Osmosis water | / | Aqua | q.s .* | | A1 |
| MICROCARE ® | Thor | PHENOXYETHANOL, | 0.87 | | A1 |
| PE | | AQUA | | | |
| MICROCARE ® | Thor | PENTYLENE GLYCOL, | 2.17 | | A1 |
| EMOLLIENT | | AQUA | | | |
| PTG | | | | | |
| CARBOPOL | Lubrizol | CARBOMER | 0.20 | | A3 |
| ETD2050 | | | | | |
| ALCASEALAN | Hakuto | ALCALIGENES | 0.02 | | A2 |
| | | POLYSACCHARIDES | | | |
| GLYCERINE | Interchimie | GLYCERIN, AQUA | 3.26 | | A4 |
| CODEX | | | | | |
| GLUCAM E20 | Lubrizol | METHYL GLUCETH-20 | 3.26 | | A4 |
| HUMECesTANT | | | | | |
| UNITAMURON ® | Induchem | BUTYLENE GLYCOL, | 5.43 | | A5 |
| H-22 | | TAMARINDUS INCA SEED | | | |
| | | GUM, PHENOXYETHANOL | | | |
| EDETA BD | BASF | DISODIUM EDTA | 0.03 | | A1 |
| SODIUM | Panréac | SODIUM HYDROXIDE | 0.03 | | A6 |
| HYDROXIDE | | | | | |
| PELLETS PRS | | | | | |
| CODEX | | | | | |
| OILY PHASE (=IF) | | | 100.00 | | B |
| DUB ININ A | Stearinerie | Isononyl Isononanoate | q.s.* | | B1 |
| | Dubois | | | | |
| DUB 810C/MB | Stearinerie | Coco-Caprylate/Caprate | 10.00 | | B2 |
| | Dubois | | | | |
| NIKKOL | NIKKO | LIMNANTHES ALBA SEED OIL, | 10 | | B2 |
| MEADOWFOAM | CHEMICALS | TOCOPHEROL | | | |
| OIL | | | | | |
| Estogel ® M | Polymer | Castor Oil/IPDI Copolymer (70- | 8 | | B1 |
| | Expert | 90%), Caprylic/Capric | | | |
| | | Triglyceride (10-30%) | | | |
| CAS-3131 | Nusil | AMODIMETHICONE | 1.5 | 0 | B3 |
| PILOT | | | | | |
| Cationic | Croda | — | 0 | 0.41 | B3 |
| polymer of | | | | | |
| formula (III)** | | | | | |
| ASL-1 TIO2 | Daito Kasei | CI77891, Aluminium hydroxide, | 38.82 | | B2 |
| CR-50 | | Sodium Lauroyl glutamate, | | | |
| | | Lysine, Magnesium chloride | | | |
| ASL-1 YELLOW | Daito Kasei | CI 77492, Sodium Lauroyl | 3.85 | | B2 |
| LL-100P | | glutamate, Lysine, Magnesium | | | |
| | | chloride | | | |
| ASL-1 RED | Daito Kasei | CI 77491, Sodium Lauroyl | 1.02 | | B2 |
| R-516P | | glutamate, Lysine, Magnesium | | | |
| | | chloride | | | |
| ASL-1 BLACK | Daito Kasei | CI 77499, Sodium Lauroyl | 0.07 | | B2 |
| BL-100P | | glutamate, Lysine, Magnesium | | | |
| | | choride | | | |

* and **: see Table in Example 2.

Preparation Protocol:

For OF: the same protocol as described in Example 1

For IF:

B1: The DUB ININ A and Estogel® M are mixed at 80° C. for 30 minutes using a dispersion blade until homogenization.

B2: In another container, the DUB 810C/MB and NIKKOL MEADOWFOAM OIL are mixed together and the four ASL pigments are added. The mixture is homogenized with a spatula (=mixture B2).

The B2 mixture is gently incorporated in mixture 1 under gentle agitation at 80° C. (=mixture X).

B3: The cationic polymer (i.e. Amodimethicone or Cationic polymer of formula (III)) is finally added to mixture X and agitation is continued for 3 minutes at 80° C.

Parameters of the Microfluidic Method:

In these tests, the following flow rates and parameters were used:

TABLE 9

| Parameters of the | Flow rate |
|---|---|
| microfluidic method | (per nozzle; in ml/h) |
| IF | 20 |
| OF | 180 |
| BF*** | 21.6 |

***Optionally, provision can be made to add a viscosity-increasing solution (BF) to the continuous phase, to improve suspending of the drops of the dispersed phase in the continuous phase, in particular such as described in WO2015055748. This solution BF in particular is a sodium hydroxide solution (NaOH).

The kinetic stability tests carried out in Example 1 (i.e. adhesion, aggregation and turbidity (or opacification) of the aqueous phase) were repeated with the dispersions C and D.

The results obtained show that, for a macroscopic dispersion provided with a dispersed phase having a high pigment content, the cationic polymer of the invention possesses the same advantageous technical effects as amodimethicone.

In addition, the dispersions C and D have similar advantageous properties firstly in terms of coverage of imperfections and colour, and secondly on the level of sensory perception, freshness, hydration and lightness.

### CONCLUSION

A cationic polymer of formula (I) therefore affords a credible, non-silicone, biosourced alternative to amodimethicone for shell formation and for stabilization of dispersions, in particular macroscopic dispersions.

1. A dispersion formed of a dispersed phase in the form of drops and a continuous phase, comprising a fatty phase and an aqueous phase non-miscible with each other at ambient temperature and atmospheric pressure, one of the phases from among the fatty phase and the aqueous phase being the dispersed phase and the other phase among the fatty phase and the aqueous phase being the continuous phase,
wherein the drops comprise at least one core and at least one shell formed of at least one anionic polymer and at least one cationic polymer, the cationic polymer being a compound having the following formula (I): 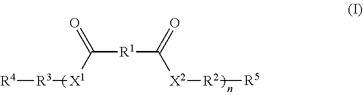
where:
n is an integer of 1 to 5, preferably of 1 to 3, more preferably of 1 or 2;
X¹ and X², the same or different, are each independently —O—, —CH₂— or —NH—, preferably at least one of the groups X¹ and X², even X¹ and X², being —NH—;
R¹, R² and R³, the same or different, are each independently a linear, cyclic or branched, divalent alkylene radical having 20 to 50 carbon atoms, preferably 25 to 45 carbon atoms, better still 30 to 40 carbon atoms, and most preferably 34 to 36 carbon atoms, R¹, R² and R³ possibly including a cycloalkylene radical having 3 to 10 carbon atoms, preferably having 6 carbon atoms, said cycloalkylene radical optionally being substituted by one or more alkyl chains having 6 to 10 carbon atoms;
R⁴ and R⁵, the same or different, are each independently H, OH or NH₂, at least one of the groups R⁴ and R⁵ being NH₂.

2. The dispersion according to claim 1, wherein the fatty phase is the dispersed phase and the aqueous phase is the continuous phase preferably in gel form.

3. The dispersion according to claim 1 or 2, wherein the cationic polymer is a lipophilic polymer able to be ionized in contact with an aqueous phase, and the anionic polymer is a hydrophilic polymer able to be ionized.

4. The dispersion according to any of the preceding claims wherein, in formula (I), X¹ and X² are —NH—.

5. The dispersion according to any of the preceding claims wherein, in formula (I), R⁴ and R⁵ are NH₂.

6. The dispersion according to any of the preceding claims, wherein the cationic polymer has the following formula (II): 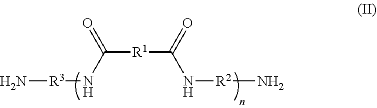
where n, R¹, R² and R³ are such as defined in claim 1, R² and R³ preferably being the same.

7. The dispersion according to any of the preceding claims, wherein the cationic polymer has the following formula (III): 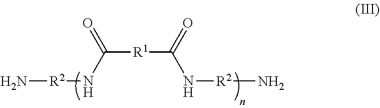
where n, R¹ and R² are such as defined in claim 1.

8. The dispersion according to any of the preceding claims, wherein each drop comprises from 0.001% to 10%, preferably from 0.005% to 5%, better still from 0.01% to 2.5%, more particularly from 0.05% to 1%, even from 0.1% to 0.5% by weight of cationic polymer(s) relative to the total weight of the phase comprising the same, and in particular the fatty phase.

9. The dispersion according to any of the preceding claims, wherein the anionic polymer is a polymer comprising monomer units comprising at least one carboxylic acid chemical function, preferably selected from among carbomers or an acrylates/C₁₀₋₃₀ alkyl acrylate Crosspolymer, and preferably a carbomer.

10. The dispersion according to any of the preceding claims, wherein said dispersion comprises from 0.01% to 10%, preferably from 0.05% to 5%, more preferably from 0.10% to 2.5%, even from 0.5% to 1% by weight of anionic polymer(s), of carbomers in particular, relative to the total weight of the phase comprising the same, even of said dispersion.

11. The dispersion according to any of the preceding claims, wherein the drops comprise a core that is liquid or at least partially gelled or at least partially thixotropic, said core being single-phase or comprising an intermediate drop of an intermediate phase and at least one, preferably only one, inner drop of an inner phase arranged in the intermediate phase, the intermediate phase and the inner phase being non-miscible with each other at ambient temperature and atmospheric pressure.

12. The dispersion according to any of the preceding claims, wherein the mean diameter of the drops of the dispersed phase is from 0.2 μm to 3 000 μm, preferably from 20 μm to 2 500 μm, in particular from 200 μm to 2 000 μm, and most preferably from 500 μm to 1 500 μm.

13. The dispersion according to any of the preceding claims, wherein the fatty phase further comprises at least one lipophilic gelling agent, preferably selected from among organic or mineral, polymeric or molecular lipophilic gelling agents; fats solid at ambient temperature and ambient pressure; and mixtures thereof, and is preferably selected from the group composed of polymeric lipophilic gelling agents, and in particular from the group composed of polyacrylates, esters of dextrin and fatty acid(s), esters of glycerol and fatty acid(s), polyamides, and mixtures thereof.

14. The dispersion according to any of the preceding claims, comprising from 0.5% to 70%, preferably from 1% to 60%, in particular from 1.5% to 50%, better still from 2% to 40%, in particular from 5% to 30%, and most preferably from 10% to 20% by weight of lipophilic gelling agent(s) relative to the total weight of the fatty phase comprising the same.

15. The dispersion according to any of the preceding claims, characterized in that it does not contain a surfactant.

16. A method for preparing a dispersion such as defined in any of the preceding claims, comprising the following steps:
optionally, heating an oily fluid FI and/or an aqueous fluid FE, to a temperature of 40° C. to 150° C.;
placing the aqueous fluid FE and the oily fluid FI in contact; and
forming fatty phase drops composed of the oily fluid FI, dispersed in a continuous aqueous phase composed of the aqueous fluid FE, or conversely, said drops comprising a shell insulating the core of the drops,
wherein:
the oily fluid FI comprises at least one oil and at least one cationic polymer of formula (I) according to claim 1, and optionally also at least one lipophilic gelling agent; and
the aqueous fluid FE at least comprises water and at least one anionic polymer, in particular a carbomer, and optionally also at least one hydrophilic texturizing agent.

17. A composition, in particular cosmetic composition, comprising at least one dispersion according to any of claims 1 to 15, optionally in association with at least one physiologically acceptable medium.

18. A non-therapeutic cosmetic treatment method, in particular make-up and/or care method, of keratin material in particular of the skin, lips or hair, comprising at least one step to apply to said keratin material at least one dispersion according to any of claims 1 to 15, or at least one composition according to claim 17.
